(19) United States
(12) Patent Application Publication
(10) Pub. No.: US 2005/0239072 A1
(43) Pub. Date: Oct. 27, 2005

(54) Device and methods for concentrating prion protein isoforms

(76) Inventors: Brian Neil Zeiler, Twp. of Washington, NJ (US); Abraham Grossman, Plesantonville, NY (US)

Correspondence Address: PERKINS, SMITH & COHEN LLP, ONE BEACON STREET, 30TH FLOOR, BOSTON, MA, 02108, US

(21) Appl. No.: 10/514,509

(22) PCT Filed: Apr. 15, 2003
(86) PCT No.: PCT/US03/11715

§ 371 Date: Nov. 12, 2004

Related U.S. Application Data
(60) Provisional application No. 60/372,556, filed on Apr. 15, 2002.
(60) Provisional application No. 60/372,548, filed on Apr. 15, 2002.

(51) Int. Cl. C12Q 1/68; C12M 1/34

(52) U.S. Cl. 435/6; 435/287.2

(57) ABSTRACT

Disclosed herein are a device and methods used to interact with proteins. In particular, a device, compositions and methods used to concentrate and detect prion proteins within a sample.

### RELATED APPLICATIONS

This patent application claims priority to and benefit of Provisional Application Ser. No. 60/372,556, filed Apr. 15, 2002, and Provisional Application Ser. No. 60/372,548, filed Apr. 15, 2002.

### FIELD OF THE INVENTION

The present invention pertains to a device, compositions and methods used to interact with proteins. In particular, this invention is directed toward a device, compositions and methods used to concentrate and detect prion proteins involved in protein agglomeration or plaque formation.

### BACKGROUND OF THE INVENTION

Several human diseases have been attributed to a unique infectious protein referred to as the prion. The prototypical human illness is Creutzfield-Jakob disease (CJD), subacute spongiform encephalopathy. Other prion illnesses of humans include kuru, Gerstmann-Straussler-Scheinker syndrome, and familial fatal insomnia. Prion-related illnesses are unique in that they may be hereditary, may occur spontaneously, or may be acquired by contamination of an agent. The appearance of variant CJD in association with the outbreak of bovine spongiform encephalopathy, postulated to be the result of contamination of beef, has greatly increased interest in a group of illnesses that are relatively rare.

This group of neurologic illnesses has been referred to by the term “slow infection,” a term introduced by Bjorn Sigurdsson in 1954 when describing scrapie, a prion illness of sheep. The characteristics of slow infections include (1) a very long period of latency lasting for several months to several years; (2) a protracted course after clinical symptoms have appeared, generally ending in death; and (3) limitation of the infection to a single host species and anatomic lesion in only organ or tissue system. Slow viral illnesses can be classified into those that are the consequence of conventional, identifiable viruses (progressive multifocal leukoencephalopathy [PML], subacute sclerosing panencephalitis [SSPE], progressive rubella encephalitis) and those associated with unconventional infectious agents, namely, prions.

The prion protein (“PrP”) generally exists as a membrane-bound sialoglycoprotein that is a normal cellular constituent distributed chiefly, although not exclusively, in the brain. Neurons, in particular, contain high concentrations of cellular PrP (“PrP^{c}”) and the protein appears to be developmentally regulated. The gene for PrP is located on the short arm of chromosome 20 in humans. Prion diseases are the result of an abnormal isoform of PrP^{c} referred to as PrP^{sc}, the scrapie isoform. Whereas PrP^{c} exists as a predominately α-helical structure, the PrP^{sc} isoform consists of a significantly increased percentage of β-pleated sheets and arises from post-translational changes in the conformation of PrP^{c}. Unlike PrP^{c}, PrP^{sc} resists proteolytic digestion and spontaneously aggregates to produce rodlike or fibrillary particles (scrapie-associated fibrils, prion rods) that can be isolated from brains of animals and humans with this class of illness.

It would be advantageous to be able to have an analytical system capable of detecting and differentiating normal, physiological PrP^{c} from pathological PrP^{sc}.

### SUMMARY OF THE INVENTION

The present invention is directed to compositions, articles of manufacture, methods, kits and devices for concentrating, and/or determining the presence or absence of prion protein in a sample.

One embodiment of the invention directed to a composition comprising a nucleic acid ligand affixed to a support. The nucleic acid ligand has affinity for prion protein. This composition is useful for concentrating prion protein putatively contained in a sample. As used herein the term sample refers to any material that one may desire to evaluate. The nature of the present invention suggests that the sample will generally have a biological nature. For example, a sample may comprise a biological fluid, or tissue of a swab. The sample may be processed to make the compounds of the sample more accessible. For example, a solid sample may be solubilized. As used herein the term “ligand” means a composition that binds or has affinity to another composition.

A preferred nucleic acid ligand is RNA. Such RNA exhibits affinity for the prion protein through non-covalent binding. That is, the RNA through folding and interaction through functional groups has a specific affinity for the prion protein similar to that of an antibody. In one aspect the RNA is RQ 11+12.

As used herein the term “support” denotes a material or things which can be separated from a solution in which it is placed. By way of example, without limitation supports may comprise particles, beads, dipsticks, fibers, filaments, inner walls of containment vessels, thin-layer plates, membranes, and gels. Particles may be magnetic or nonmagnetic. In one aspect the support is a membrane.

In one embodiment, the compositions of the present invention preferably bind prion protein that is insoluble. Thus, compositions of the present invention can be used to distinguish between different isoforms of the prion protein. However, where the composition binds both isoforms, the different isoforms can be distinguished by resistance to enzyme digestion. For example, isoforms that are resistant to proteinase K digestion are typically associated with disease states.

The compositions of the present invention can be incorporated in devices for isolating prion protein. One embodiment of the present invention features a device comprising a vessel for containing a sample and a support. The support has a nucleic acid ligand affixed thereto. The nucleic acid ligand has affinity to prion protein such that sample putatively containing prion protein can be placed in contact with the vessel and the prion protein, if present in the sample, binds to said nucleic acid ligand and is immobilized on the support. The support can be separated from the solution after the prion protein is bound to the nucleic acid ligand.

Preferably, the vessel has an inlet and an outlet to allow sample to flow into the vessel through the inlet and allow said sample to flow out of the vessel through the outlet. For example, the vessel can comprise a cartridge or column which contains the nucleic acid ligand on a support. A cartridge or column allows a substantially continuous flow of sample during the period in which the prion protein is being bound to the nucleic acid ligand. Cartridges and columns normally comprise a substantially cylindrical housing having two open ends. One end, an inlet opening, receives sample. One end, an exit opening, discharges effluent and/or eluate. The cylidrical housing forms a chamber for containing a support having the nucleic acid ligand and sample.

The devices and compositions of the present invention are incorporated in kits. As used herein kits are assemblies of devices, compositions, and articles of manufacture packaged with instructions for their use. One kit of the present invention, for concentrating prion protein in a sample, comprises a nucleic acid ligand affixed to a support with instructions for its use. The nucleic acid ligand has affinity for prion protein. The instructions instruct the user to place a sample in the presence of the support under binding conditions to form an immobilized prion protein. As used herein, the term “binding conditions” refer to those conditions in which the nucleic acid ligand exhibits affinity toward the prion protein.

The support can be selected from the group consisting of particles, beads, dipsticks, fibers, inner walls of containment vessels, thin-layer plates, membranes, and gels, for the convenience of the user. In one aspect, the support is contained in a device such as a cartridge or column.

The kit preferably has reagents and instructions to facilitate the separation and or detection of different isoforms of prion protein. Wherein prion protein bound to said nucleic acid ligand is insoluble, the kit, preferably, has enzyme activities which are capable of digesting the soluble isoform. Thus, prion protein remaining after enzyme digestion suggests the presence of the insoluble isoform. The kit preferably includes instructions for the subjecting the prion protein concentrated on the support to enzyme digestion to form digested protein or surviving, insoluble protein. One such enzyme is proteinase K.

The surviving protein can be identified with one or more nucleic acid ligands having affinity for the prion protein. In one aspect, the ligand is labeled in a manner known in the art such that any labeled ligand bound to the prion protein can be readily identified. In one aspect, the kit further comprises one or more antibodies capable of binding an insoluble isoform.

Embodiments of the present invention are directed to methods of concentrating prion protein comprising the steps of providing a nucleic acid ligand affixed to a support in which the nucleic acid ligand has affinity for prion protein. Next, the method comprises the step of placing a sample in contact with said support under conditions in which said nucleic acid and prion protein form an affinity complex.

Further methods of the present invention distinguish the isoforms of the prion protein by steps of digesting the prion protein affixed to the support to form a digestion product. Preferably, the method includes the step of placing a second ligand in the presence of the digestion product to bind to prion protein if present. The presence of prion protein in the digestion product suggests the presence of the insoluble form of prion protein.

### BRIEF DESCRIPTION OF THE DRAWINGS

FIG. 1 depicts a composition of matter or article of manufacture in accordance with the present invention;

FIG. 2 depicts a device, in cross section, made in accordance with the present invention;

FIG. 3 depicts a flow diagram setting forth steps in a method of distinguishing isoform of the prion protein;

FIG. 4(a) is a schematic of the cartridge use, (b) is hrPrP from serum, (c) is hrPrP from urine, (d) is PrP^{Sc} from serum, and (e) is PrP^{Sc} from urine; and

FIG. 5(a) is a Western blot analysis using mAb 3F-4; (b) is a Western blot of bound material removed from the RQAAA adsorbent, and (c) is a Western blot analysis with monoclonal antibodies 7A12.

### DETAILED DESCRIPTION

The present invention will be described in detail as compositions, methods, kits and devices for concentrating, and/or determining the presence or absence of prion protein in a sample. However, individuals skilled in art will recognize that features of the present invention have application for the detection of other materials. Embodiments of the present invention have therapeutic, diagnostic, forensic and analytical applications.

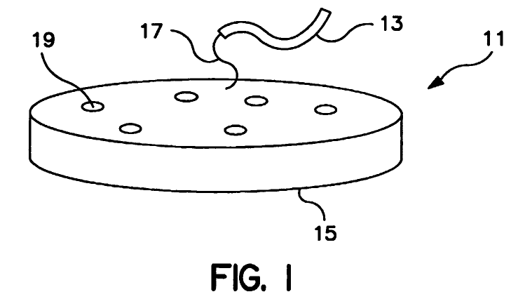

One embodiment of the invention directed to an article of manufacture or composition is depicted in FIG. 1. The composition or article of manufacture is generally designated by the numeral 11. The composition or article of manufacture comprises a nucleic acid ligand 13 affixed to a support 15. The nucleic acid ligand having affinity for prion protein. The term “composition or article of manufacture” is used because the invention is not limited as to the nature of the support. That is, the support may take many forms, some forms may be of such a small scale that the word device or article of manufacture may seem inappropriate.

The article of manufacture or composition 11 is useful for concentrating prion protein putatively contained in a sample. As used herein the term “sample” refers to any material that one may desire to evaluate. The nature of the present invention suggests that the sample will generally have a biological nature. For example, a sample may comprise a biological fluid, or tissue of a swab. The sample may be processed to make the compounds of the sample more accessible. For example, a solid sample may be solubilized. In processing a sample, care must be taken so as not to destroy the nature of the target molecule. The article of manufacture or composition 11 of the present invention can be used to concentrate insoluble prion protein after digestion with protein digestion enzymes. A preferred biological sample is cerebral spinal fluid, urine, blood, serum, plasma, and brain tissue extracts.

The term “concentrating” is used to mean isolating, separating or bringing together in one location.

In one aspect, the nucleic acid ligand is an RNA. Such RNA exhibits affinity for the prion protein through non-covalent binding. That is, the RNA through folding and interaction through functional groups has a specific affinity for the prion protein similar to that of an antibody. In one aspect, the RNA is RQ 11+12. The nucleic acid sequence for RQ 11+12 is set forth in SEQ ID NO. 1.

5′GGGGUUUCCAACCGGAAUUUGAGGGAUGCCUAGGCAUCCCCCGUGCG
UCCCUUUACGAGGGAUUGUCGACUCUAGUCGACGUGUGGGCGAAAAAU
GUACGAGAGGACCUUUUCGGUACAGACGGUACCUGAGGGAUGCCUAGG
CAUCCCCGCGCGCCGGUUUCGGACCUCCAGUGCGUGUUACCGCACUGUC
GACCC 3′

It is believed that RNAs with features relating to viral protein coats will capture prion protein. And, in particular retroviral protein coats.

As illustrated, support 15 is a membrane having openings 19 through which fluids may flow. However, supports may comprise particles, beads, dipsticks, fibers, filaments, inner walls of containment vessels, thin-layer plates, membranes, and gels. Particles may be magnetic or nonmagnetic. Methods of associating a nucleic acid with a support are well known in the art. The composition or article of manufacture is illustrated with a chemical linking group 17 that makes the nucleic acid more accessible.

Preferably, the composition or article of manufacture 11 binds one of the isoforms preferentially, and, in one aspect, the isoform that is insoluble. However, where this not the case, the compositions and articles of manufacture 11 of the present invention can be used to distinguish between different isoforms of the prion protein. Where the composition or article 11 binds both isoforms, the different isoforms can be distinguished by resistance to enzyme digestion. Soluble isoforms generally are susceptible to digestion. Isoforms that are resistant to digestion are insoluble. Proteinase K digestion is used in the art to distinguish between isoforms. Insoluble isoforms are typically associated with disease states. Conditions for making a digestion product in the presence of a composition or article of manufacture 11 are well known to those skilled in the art.

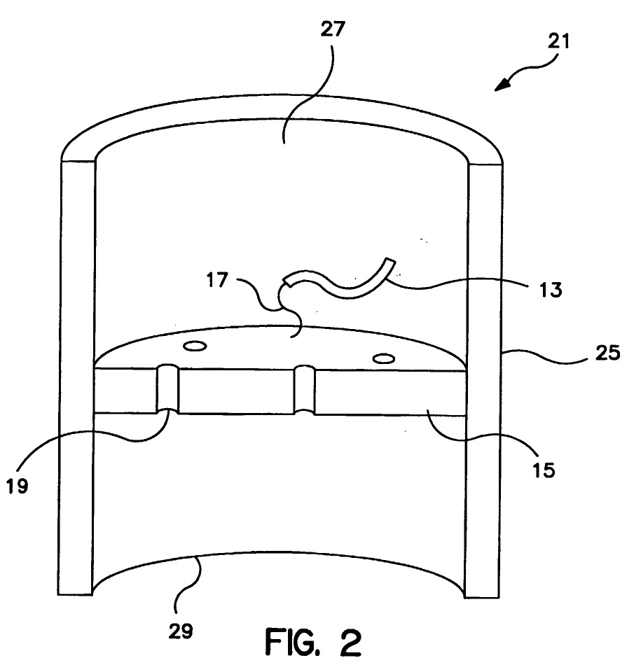

The compositions or articles of manufacture 11 of the present invention can be incorporated in devices for concentrating prion protein. Turning now to FIG. 2, one embodiment of the present invention features a device, generally designated by the numeral 21, for concentrating prion protein. The device 21 comprising a vessel 25 for containing a sample [not shown] and a support in the form of a membrane 15. The support 15 has a nucleic acid ligand 13 affixed thereto. The nucleic acid ligand 13 has affinity to prion protein such that sample potentially containing prion protein may be placed in the vessel 25. Prion protein, if present, binds to the nucleic acid ligand 13 and is immobilized one the support 15. The support 15 can be separated from the solution after the prion protein is bound to the nucleic acid ligand 13.

As illustrated, the vessel 25 has an inlet end 27 and an outlet 29. Sample flows into the vessel 25 through the inlet end 27, through support 15 openings 19, and flows out of the vessel 25 through the outlet end 29. The vessel 25 is a cartridge or column configuration. A cartridge or column allows a substantially continuous flow of sample during the period in which the prion protein is being bound to the nucleic acid ligand 13. The cylindrical housing forming vessel 25 forms a chamber 31 for containing a support 15 having the nucleic acid ligand 13 and sample. In the event the support, were beads or particles the vessel 25 may be equipped with frits or screens [not shown] to retain the support.

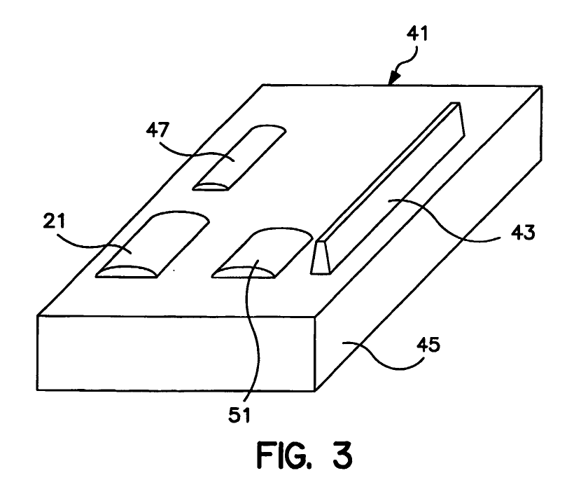

The devices, articles of manufacture and compositions of the present invention can be incorporated in kits. Turning now to FIG. 3, a kit, generally designated by the numeral 41, embodying features of the present invention, is depicted. The kit 41, for concentrating prion protein in a sample, comprises a nucleic acid ligand 13 affixed to a support 15 held in a device 21 with instructions 43 for its use. The instructions 43 instruct the user to place a sample in the presence of the support 13 under binding conditions to form an immobilized prion protein.

Kit 41 is illustrated with packaging in the form of a box 45 for holding the parts in a bundle for the convenience of the user. Other packaging materials and forms are also well understood in the art.

As illustrated the kit 41 has reagents and instructions to facilitate the separation and or detection of different isoforms of the prion protein. Wherein prion protein bound to said nucleic acid ligand 13 is insoluble, the kit, preferably, has enzymes which are capable of digesting the soluble isoform. Thus, prion protein remaining after enzyme digestion suggests the presence of the insoluble isoform. The kit preferably includes instructions for the subjecting the prion protein concentrated on the support to enzyme digestion to form digested protein or surviving, insoluble protein. A preferred enzyme is proteinase K which is depicted as a first vial 47.

The surviving protein can be identified with one or more ligands having affinity for the prion protein. Preferably, the ligand is labeled in a manner known in the art such that such ligand bound to the prion protein can be readily identified. A preferred ligand in a nucleic acid ligand or an antibody. Preferably, the kit 41 further comprises one or more antibodies capable of binding an insoluble isoform. A preferred antibody is selected from the group comprising mAb 3F-4, 7A12 and alike. The antibodies are depicted as second vial 51.

The instructions 43 describe the operation of the kit 41, the device 25 and the composition or article of manufacture 11. The instruction 43 would instruct the user to use the nucleic acid ligand 13 affixed to the support 15 to immobilize prion protein. The user would be instructed to flow sample through vessel 25 under binding conditions to allow the nucleic acid ligand and prion protein form an affinity complex. Preferably, the binding conditions would be set forth in the instructions.

The instructions 43 would describe steps to distinguish the isoforms of the prion protein. The steps include digesting the prion protein affixed to the support 15 to form a digestion product. Preferably, the method includes the step of placing a second ligand in the presence of the digestion product to bind to prion protein, if present. The presence of prion protein in the digestion product suggests the presence of the insoluble form of prion protein.

These features and others will be apparent from the following Examples.

### EXAMPLE

To examine if RNA could be used to bind PrP proteins from biological solutions, a column cartridge containing an adsorbent impregnated with RNA was developed and subjected to analysis.

Enzymes and Reagents. Transcription reagents and high purity BSA were purchased from Ambion (Austin, Tex.) or MBI Fermentas (Hanover, Md.). Schleicher and Scheull 0.45 μM BA85 nitrocellulose membranes were used (VWR) and PVDF and Nylon membranes and radioisotopes were purchased from Perkin Elmer (Boston, Mass.).

Purification of PrP Proteins and Extracts. Recombinant human PrP²³⁻²³¹, PrP²³⁻¹⁴⁴, PrP⁹⁰⁻²³¹ were kindly provided by Man-Sun Sy, Case Western University, and were purified to homogeneity from E. coli as GST tagged fusions (with removal of GST by thrombin cleavage) as described previously by Weiss, S. et al., J. Virology 69(8), (1995) pp. 4776-4783, the entire teachings of which are incorporated herein by reference. Ten percent (wt/vol) brain homogenates from wildtype mice were prepared as follows. Whole brains (Psychogenics, Inc., New York) were homogenized in nine volumes of PBS supplemented with 0.5% Nonidet P-40, 0.5% deoxycholic acid and Protease Inhibitor Cocktail for Use with Mammalian Cell and Tissue Extracts (Sigma). After centrifugation (12,000×g for 30 minutes), the supernatants were aliquoted and stored at −70° C. Protein concentration was measured by Bradford assay, using Pierce reagents (VWR). Semi-purified scrapie extracts from infected mice were the kind gift of Dr. Richard Rubenstein and Dr. Richard Kaszsak prepared as previously described by Li, R., et al., J. Mol. Bio. 301(3), (2000) pp. 567-573, the entire teachings of which are incorporated herein by reference.

Construction of RNAs. RNAs were synthesized in vitro using T7 RNA polymerase with PCR-generated DNA templates. DNA templates for PCR were either sequenced plasmid constructs in pUC18/19 or synthetic oligonucleotides using additional 5′ primer sequence to add the 17 RNA polymerase promoter. [alpha-³²P] CTP was used for internal labeling of RNAs. All of the RNAs used in this study were gel purified from 7 M urea gels and eluted with a V-channel electroelution apparatus (IBI). RNA concentrations were determined for labeled RNAs as a function of isotope incorporation using a BioScan XER-2000 or for unlabeled RNAs by a RiboGreen (Molecular Probes) fluorescence-binding assay using a Sequoia-Turner Model 450 flourometer. The secondary structures were created with RNAdraw, see Matzura, O. et al., Computer Applic. Biosci. 12(3), (1996) pp. 247-249, the entire teachings of which are incorporated herein by reference.

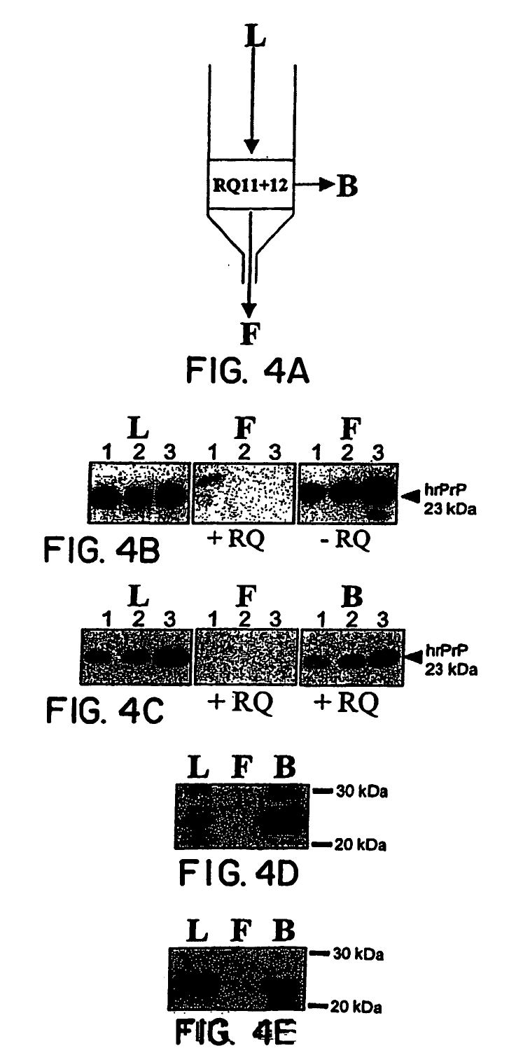

In this example, RQ11+12 was the nucleic acid ligand employed as a representative RNA since RQ11+12 demonstrates high affinity and specific binding to hrPrP. However, other nucleic acid ligands could have been used as well. RQ11+12 was synthesized with a 15-nucleotide poly-A tail (RQAAA) and then hybridized to oligo-dT-cellulose beads using methods well known in the art. The slurry was poured into a filter column containing a 0.2 μM membrane that is suitable for a volume of approximately 1 mL sample. Multiple loadings allowed for the examination of at least 10 mL of sample solution. Samples of the material loaded onto the column (L), retrieved from the RQAAA adsorbent (B) and the material that flowed through the column (F) were examined by Western Blot analysis, see FIG. 4.

FIG. 4(a) is a schematic of the cartridge use, wherein L is the material loaded onto cartridge, F is the material that flowed through cartridge, and B is the material bound by adsorbent followed by removal for analysis; (b) is hrPrP from serum wherein the left panel contains a sample of the material loaded onto the columns (L), wherein the middle panel contains the flow-through (F), removed in a 1 mL volume, for a cartridge with RQAAA, and wherein the right panel contains the flow-through (F) from a cartridge without RQAAA; (c) is hrPrP from urine wherein Lane 1 is 500 ng/μL hrPrP, lane 2 is 1500 ng/μL hrPrP, lane 3 is 3000 ng/μL hrPrP. Ten μL were analyzed by western blot. 49 pmoles of RQAAA bound at least 3 ug hrPrP (105 pmoles); (d) is PrP^{Sc} from serum 25 μL adsorbent was used for filtering 10 mL of serum spiked with PrP^{Sc} scrapie extract; and (e) is PrP^{Sc} from urine wherein 25 μL columns were used for filtering 10 mL of urine spiked with PrP^{Sc} scrapie extract.

The RNA cartridges demonstrated the ability to effectively bind hrPrP from serum and urine. Solutions containing 10% bovine calf serum were spiked with increasing amounts of hrPrP and loaded onto the column to test for binding to adsorbent with or without RQAAA (see FIG. 4b). Western blot analysis of the material that flowed through the column clearly indicated that RNA was necessary for the filtration effect. When the adsorbent was impregnated with RNA, there were no detectable amounts of hrPrP in the flow through (F; middle panel), indicating efficient binding to the column cartridge. However, if RQAAA was omitted from the adsorbent, virtually all of the hrPrP flowed through the column (right panel). These results demonstrate that RQAAA RNA is responsible for the PrP binding activity of the cartridge.

The cartridge was also effective for binding hrPrP from urine, see FIG. 4c. Increasing amounts of hrPrP were spiked into a solution containing 10% urine and passed through a column cartridge containing the RQAAA adsorbent. A clear signal is generated from 10 μL of sample loaded onto the column (L; left panel). There were no detectable amounts of hrPrP in 10 μL of flow through (F; middle panel), as apparently all of the detectable material was bound to the adsorbent (B; right panel).

The RNA column cartridge was also tested for binding PrP^{Sc} derived from scrapie-infected mice. Bovine calf serum spiked with scrapie extract was passed through the column, analyzed by western blot, and PrP^{Sc} could be detected in the loaded (L) and bound (B) fractions, but not in the flow through (F), see FIG. 4d. Similar data was obtained when human urine was spiked with the same scrapie extract—easily detectable signals were obtained in the loaded (L) and bound (B) fractions, see FIG. 4e. In this experiment, however, a small trace signal can be observed in the flow-through fraction (F), perhaps due to a slight decrease in affinity for the PrP^{Sc} isoform. Extracts from scrapie-infected hamster gave similar results (data not shown). Significantly, extensive proteinase K digestion was used in the preparation of the scrapie extract to remove the protease sensitive PrP^{C} isoform. Thus, RNA adsorbent bound PrP^{Sc} from proteinase K treated biological samples, warranting further investigation of the practical applications of this technology.

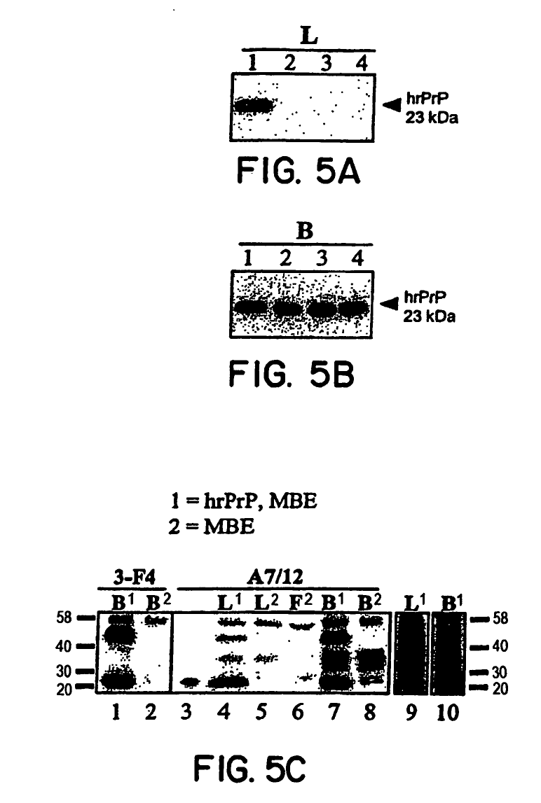

RNA cartridges increase the limits of detection of PrP 1000-fold. It is possible to load 10 mL of sample onto a single PrP-column and remove the material bound to the adsorbent in volumes as small as 10 μL; therefore, the RNA column should be able to serve as a PrP-concentration device. Four solutions of 10% serum were prepared with decreasing concentrations of spiked hrPrP, ranging from 1 ng/μL to 1 pg/μL. Ten microliters of each solution were analyzed by western blot prior to passage through RQ11+12 adsorbent, see FIG. 5a. At these concentrations, a signal could be detected only at the highest concentration, representing a total mass of 10 ng of hrPrP, which is near the detection limit expected using chemiluminescent Western blotting techniques. Amounts of 1 ng PrP or less were undetectable by Western Blot. A volume that correlates to a total mass of 10 ng PrP for each of the four solutions was passed through the column. Material bound to the adsorbent was removed in a 10 μL volume and examined by Western Blot, see FIG. 5b. The material from all four solutions produced a signal equivalent in intensity to 10 ng of starting material. The RNA cartridge enabled the detection of hrPrP at concentrations that were previously undetectable. This approach effectively increased the level of sensitivity of western blot analysis 1000-fold.

FIG. 5(a) is a Western blot analysis (mAb 3F-4) of samples loaded into the cartridge; and (b) is bound material removed from the RQAAA adsorbent. The concentration of hrPrP in the solution loaded into the cartridge was 1 ng/μL (lane 1), 0.1 ng/μL (lane 2), 0.01 ng/μL (lane 3), 0.001 ng/μL (lane 4). Increasing volumes of sample were added such that a total of 10 ng were loaded onto the column per sample (10 μL in lane 1, 100 μL in lane 2, 1 mL in lane 3, and 10 mL in lane 4). After binding, hrPrP was removed from the adsorbent in a volume of 10 μL that was examined by western blot analysis with the monoclonal antibody 3F-4; and (c) is a Western blot analysis with monoclonal antibodies 7A12 (mouse, human; lanes 1-6) or 3F-4 (human; lanes 7-8). Solution 1 was 2 ng/μL hrPrP, 1.6 μg/μL MBE and Solution 2 was 1.6 μg/μl MBE only. Lane 1 and 7 are from (b) (removed in a volume of 10 μL) from 200 μL Solution 1; Lanes 2 and 8 is from (b) a 200 μL Solution 2; Lane 3 is 10 ng hrPrP (in 10% BCS); Lane 4 is 15 μL Solution 1 (L; 30 ng hrPrP, 24 μg MBE); Lane 5 is 15 μL Solution 2 (L; 24 μg MBE); Lane 6 is 15 μL F from 200 μL Solution 2. Lanes 9 and 10 are Coomasie stained; Lane 9 is 10 μL Solution 1 (L); lane 10 is 10 μL Solution 1 (B). L is material loaded onto cartridge; F is material that flowed through cartridge; B is material bound by adsorbent followed by removal for analysis; MBE—mouse brain extract.

The ability of the RQ11+12 adsorbent to increase the sensitivity of western blot analysis was applied to detect endogenous PrP^{C} directly from mouse brain homogenates, see FIG. 5c. Two test solutions containing mouse brain extract (MBE; 1.6 μg/μL) were prepared; a control solution spiked with 2 ng/μL hrPrP (Solution 1) and a test solution containing just MBE (Solution 2). Four hundred microliters of each solution were passed through columns containing the RQAAA adsorbent and analyzed as above. A sample of Solution 1 removed from the column (B¹; lane 1) produced two major bands corresponding to hrPrP monomer (23 kDa; compare lanes 1 and 3) and dimer (˜50 kDa), which we routinely observe at higher concentrations (>20 ng/sample). A sample of Solution 2 removed from the column (B²; lane 2) produced a single band of approximately 60 kDa that we accredit to an immune reactive, non-specific component present in MBE that weakly binds to the RNA column. This background signal is present in all samples containing MBE, irrespective of mAb. The absence of signal around 35 kDa, the expected size for native, diglycosylated PrP^{C} (see Caughey, B. et al., J. Viorology 63(1), (1989) pp. 175-181, the entire teaching of which is incorporated herein by reference), is not surprising because mAb 3F-4 does not bind to mouse PrP^{C} (see Kascsak, R. et al., Immunol. Invest. 26(1-2), (1997) pp. 259-268, the entire teaching of which is incorporated herein by reference).

To detect the presence of endogenous mouse PrP^{C} (mPrP), the above solutions were also examined with the mAb 7A12, which can bind to both hrPrP and mPrP (see Li, R. et al., J. Mol. Bio. 301(30, (2000) pp. 567-573, the entire teaching of which is incorporated herein by reference). A 15 μL sample of Solution 1 that was loaded onto the column (L¹; FIG. 5c, lane 4) generated the same banding pattern as observed in lane 1 with the addition of a faint band at 35 kDa due to the presence of endogenous mPrP. This signal is greatly enhanced in the material removed from the column (B¹; lane 7), demonstrating the effectiveness of the RNA column in increasing the signal from endogenous mPrP. The same treatment with Solution 2 also demonstrates a tremendous increase in signal for the 35 kDa representing endogenous mPrP (compare L² and B²; lanes 5 and 8). A faint doublet is also visible (B²), most likely attributable to 25 kDa and 27 kDa forms of PrP^{C} known to occur from partial enzymatic degradation (see Somerville, R. et al., Gen. Virol. 71(Pt 4), (1990) pp. 833-839, and Zanusso, G. et al., Proc. Nat. Acad. Sci. USA 95(15), (1998) pp. 8812-16, the entire teachings of which are incorporated herein by reference). These truncated forms of PrP^{C} appear to have a lower affinity for the column because their signal does not increase dramatically (compare L² and B²; lanes 5 and 8) nor bind fully to the RNA column (compare L² and F²; lanes 5 and 6). Coomasie staining of 10 μL samples from Control Solution 1 reveal that the column retains some amount of MBE bulk protein, however, it appears that enrichment is mostly for PrP proteins (lanes 9 and 10). Taken together, these data clearly demonstrate the effectiveness of the RQAAA column in increasing the sensitivity of Western detection of PrP from biological solutions.

The RNAs that make up this collection are meant to represent a diversity of secondary structures. They are derived from company projects to engineer RNAs with affinity to epitopes of interest and the ability to be amplified by Q-beta replicase. (See Zeiler, B. et al., Proceedings of SPIE Aerosense 2000, 4036, (2000) pp. 103-114, the entire teachings of which are incorporated herein by reference.) The small RNAs AP1, AP46, AP49 and BS1577 are not amplifiable. AP1 (29 nt; 5′GGGAAUUUGAGGGACG AUGGGUAAGUGGG 3′, SEQ ID NO. 2) was isolated by using SELEX technology. AP46 is AP1 without the bases in bold face, and AP49 is AP1 with the underlined bases replaced with adenine. BS1577 (56 nt; 5′ GGGCCCCGUAACUUCGGGAGAAGG GGUGCUCUGUUAGGGUGCAAGCCCGAGAGAGC 3′, SEQ ID NO. 3) contains the 1577 region from Bacillus subtilis 23S rRNA, see Ash, C. et al., FEMS Microbiology Letters 94 (1992) pp. 75-80, the entire teachings of which are incorporated herein by reference. MDV and MNV are templates for amplification by Q-beta replicase whose sequences and structures have been previously characterized, see Preuss, R. et al., J. Mol. Bio. 273(3), (1997) pp. 600-613, the entire teachings of which are incorporated herein by reference. Other RNAs were made by inserting sequences (underlined) into MNV like MNV:AP1, which contains the AP1 sequence (italicized) (130 nt; 5′ GGGUUCAUA GCCUAUUCGGCUUCGCGCAUGGGAAUUUGAGGGACGAUGGGGAAGUGG GAGCGCGUUUUAAAGGACCUUUUUCCCUCGCGUAGCUAGCUACGCG AGGUGACCCCCCGAAGGGGGGUGCCCC 3′, SEQ ID NO. 4), MNVLO (118 nt; 5′GGGUUCAUAGCCUAUUCGGCUCGCGCCCCUGGGGUUUGCCUCA GGAGCGCGUUUUAAAGGACCUUUUUCCCUUGCGUAGUAGCUACGCGAG GUGACCCCCCGAAGGGGGGUGCCCC 3′, SEQ ID NO. 5) and MNVUP (118 nt; GGGUUCAUAGCCUAUUCGGCUUCGCGCCCGUUUAUAAUACUUAGUGAG C GCGUUUUAAAGGACCUUUUUCCCUCGCGUAGCUAGCUACGCGAGGUGA C CCCCCGAAGGGGGGUGCCCC 3′, SEQ ID NO. 6). RQT157 is another amplifiable template RNA (157 nt; 5′ GGGGUUUCGAACCGGAAUUUGAGGGAUGCCUAGGC AUCCCCCGUGCGUCCCUUUACGAGGGAUUGUCGACUCUAGAGGAUCCG G UACCUGAGGGAUGCCUAGGCAUCCCCGCGCGCCGGUUUCGGACCUCCAG UGCGUGUUACCGCACUGUCGACCC 3′, SEQ ID NO. 7) into which the Rev Responsive Element (RRE; underlined) from HIV-1 (see Iwai, S. et al. Nucleic Acids Res. 20(24), (1992) pp. 6465-6472, the entire teachings of which are incorporated herein by reference), and the Sarcin/Ricin cleavage domain (S/R; italicized, see Endo, Y. et al., J. Biol. Chem. 257(15), (1982) pp. 9054-9069, the entire teachings of which are incorporated herein by reference) were cloned, creating RQ11+12.

Filter Binding Assays. The binding affinity of hrPrP for various RNA ligands was determined using a two-filter binding assay (see Battle, D. et al., RNA 7, (2001) pp. 123-132, and Lochrie, M. et al., Nucleic Acids Res. 25(14), (1997) pp. 2902-2910, the entire teachings of which are incorporated herein by reference). Internally labeled RNAs (always at a concentration at least ten-fold lower than the protein concentration) were incubated with increasing amounts of hrPrP in 20 μL-100 μL reactions in Binding Buffer (10 mM Tris-OAc, pH 7.5, 2 mM MgCl₂, 250 mM NaCl, 1 μg/μL BSA, 2 mM DTT) unless otherwise indicated. A 100 mM NaCl Binding Buffer (10 mM Tris-OAc, pH 7.5, 100 mM NaCl, 1 μg/μL BSA, 2 mM DTT) was used where indicated. Transfer RNA was present at a concentration of 10 ng/μL (˜400 nM) where indicated. In competition assays, competitor (unlabeled) RNAs were pre-incubated with hrPrP for 15 minutes prior to the addition of labeled RNAs. After 30 minutes at 37° C., reactions were vacuum filtered in a Minifold II Slot Blot hybridization apparatus (Schleicher and Schuell) first through a PVDF or nitrocellulose filter to capture protein and RNA-bound proteins, and then through a positively-charged nylon filter to capture free RNA. Filters were washed with 400 μL 10 mM Tris-OAc, pH 7.5, 50 mM NaCl. A reaction with no protein was used to determine background. The percentage of RNA bound was determined using a Storm 820 Phosphorlmager scanner and ImageQuant software (Molecular Dynamics) by dividing the intensity of the signal on the protein-binding filter by the sum of the intensity of signals from both filters. Apparent dissociation constants (K_{d}) were determined from an average of at least three measurements at each hrPrP concentration, with an average standard deviation below 2%. The percentage of RNA bound to protein was plotted against the concentration of total protein. The apparent K_{d} is defined as the protein concentration at which 50% of maximal RNA binding occurs.

Filtration Cartridges. Fifty milligrams of oligo-(dT)-cellulose beads (Sigma) and 15 pmoles of gel-purified RQAAA RNA (RQ11+12 with 15 adenosines added to the 3′ end during transcription) were incubated in Buffer I (10 mM Tris-HCl, pH 7.5, 500 mM NaCl, 0.5% SDS) for 30 minutes at 25° C. The solution was then poured into a Nanosep MF 0.2 μM filtration column (Pall). Cartridges prepared by this method were able to be stored for at least 4 months at 4° C. with no detectable loss in activity (data not shown). After a brief spin, the column cartridge was then washed with ten volumes of Buffer II (10 mM HEPES, pH 8.0, 100 mM LiCl, 0.1 mM EDTA, 1 mM DTT). Samples were loaded onto cartridges, incubated 30 minutes at 25° C., and washed with 1 mL Buffer II. (The investigators routinely added 10 mL of solution to the cartridges, although the maximum volume that could be added was not determined.) After washing, fractions were collected (L—loaded material; B—bound material; F—flow through) for standard western analysis using 4-20% gradient gels were used (Novex). Material was removed from the column (B) by boiling in sample buffer (2 mM DTT, 0.5% SDS) and collected in a volume of either 10 μL or 1 mL. The antibodies used in this study were either mAb 3F4, which is directed against amino acids 109-113 and is reactive to feline, hamster and human PrP (see Kascsak, R. et al., Immunol. Invest. 26(1-2), (1997) pp. 259-268, the entire teachings of which are incorporated herein by reference), or mAb 7A12, which is directed against a non-linear epitope between amino acids 90-145 and is reactive to mouse and human PrP (see Li, R. et al., supra).

This study characterizes the interactions between different forms of PrP from various sources and several RNA constructs. The study reveals a number of RNA binding characteristics for hrPrP that suggest possible functions for RNA in prion biochemistry. Human recombinant PrP, hrPrP, demonstrated the ability to bind to individual RNA species with high affinity (K_{d}<100 nM), regardless of sequence or predicted secondary structure, provided that no additional RNA species were present—termed “non-specific binding.” In contrast, the term “specific binding” describes the binding activity of hrPrP to the few RNAs (RQ11+12, AP1, and MNV:AP1) whose affinity was relatively unchanged in the presence of excess competitor RNAs. The data suggests that PrP possesses two discrete RNA binding activities—a “non-specific” activity in the N-terminus (between amino acids 23-90) and a “specific activity” in the core of the protein. At least in the case of hrPrP, non-specific binding appears to be prerequisite to specific binding because RQ11+12 does not bind to truncated hrPrP lacking the N-terminal amino acids 23-90 (PrPc). The effect of the N-terminal sequence may be kinetic. Just as DNA-binding regulatory proteins first bind DNA non-specifically and then rapidly translocate across the DNA to a specific binding site, the N-terminus of hrPrP binds RNA non-specifically, rapidly bringing the bound RNA into close proximity to the proposed binding site in the core of the protein. Conditions were determined where RQ11+12 can induce pK-resistance in hrPrP at physiologic pH (unpublished work; Adler, V., Zeiler, B., Kryukov, V., Kascsak, R., Rubinstein, R., and Grossman, A.), leading to the proposal that after RQ11+12 binds to the N-terminus, a structural change occurs in hrPrP. This model is supported by the observation that N-terminal amino acids can influence the conformation of the C-terminal domain of PrP and that RQAAA can bind to PrP^{Sc}, which has had the N-terminus removed by pK-digestion and which has a different conformation (β-sheet rich) than hrPrP. Presumably, the binding site in the core of the protein is available in the PrP^{Sc} conformation.

It is likely that elements possessed by RQ11+12 and AP1 allow the formation of additional contacts with hrPrP that lead to specific binding. While RQ11+12 and AP1 have no obvious similarities, both RNAs are predicted to contain non-Watson-Crick base pairs. These base pairs in RQ11+12 are in the stem that differentiates it from the weaker binding RNA, RQT157 (see Table 1). In addition to providing increased thermodynamic stability, non-canonical base pairs can alter the three-dimensional structure of an RNA by widening the major groove, enabling interactions with extended protein domains that may play a role in the specificity of protein/RNA binding. The double stranded stem of the RNA might act as a scaffold for the binding of multiple PrP molecules, as has been proposed for dsDNA/PrP complexes formed in vitro. The ability of RQ11+12 to bind more than a single PrP peptide is demonstrated by the column cartridge where the stoichiometry of binding is greater than 2:1.

TABLE 1

| | Column I | Column II | |
|---|---|---|---|
| | Kd (nM) | Kd (nM) | Fold effect |
| RNA | (−tRNA) | (30 tRNA) | tRNA |
| MNVAPI (130 nt) | 3.8 | 12 | 3.2 |
| API (29 nt) | 5.0 | 81 | 16 |
| RQ 11 + 12 (197 nt) | 26 | 91 | 3.5 |
| AP46 (26 nt) | 14 | 100 | 7.1 |
| AP49 (29 nt) | 15 | 161 | 11 |
| MDV (244 nt) | 7.6 | 170 | 22 |
| RQT157 (157 nt) | 56 | 185 | 3.3 |
| MINVUP (I 18 nt) | 5.0 | 220 | 44 |
| MN-V (86 nt) | 38 | 1000 | 26 |
| N1NVLO(I 18nt) | 4.4 | 1700 | 390 |
| BS1577 (56 nt) | 31 | 2500 | 81 |

Continuing advances in the field of prion diagnostics can eventually lead to the detection of the disease in live animals. Early detection of prion diseases is the key issue in controlling the disease, and new assays like the luminescence immunoassay, which can detect PrP in solution at a 1 pg/ml, are very promising. Another technique, used to monitor plasma processing, uses a special protocol for the preparation of sample material followed by western blot analysis and can reportedly detect as little as ˜2500 infectious units/mL. If these techniques were combined with the RNA-based PrP-column concentrator, which can increase the level of detection by 1000-fold, it is conceivable that as little as 3 IU/mL could be detected. Recent studies have shown that PrP^{Res}, an isoform that is resistant to proteinase K digestion, is present in the urine of prion-infected animals. Although it is difficult to obtain, urine collected from captive animals may represent a good source of sample material because it is non-invasive, can be collected in large volumes and is suitable for the RQ11+12 RNA-column presented in this study. Saliva is another biological fluid that may be suitable for TSE detection because prion has been detected in the tonsils of sheep and deer. The application of RNA-based affinity concentration technology during sample preparation may prove to be very useful in the development of pre-mortem, prion diagnostics.

This study reveals the importance of RNA structure on interactions with hrPrP and demonstrates the utility of RNAs in affinity chromatography. The technology is far reaching and has the potential to be applied to situations where RNA is known to specifically bind a protein. In addition, RNA constructs that can recognize specific protein epitopes and be amplified by Q-beta replicase, such as RQ11+12, could represent a major advance in diagnostics, combining the specificity of traditional immunodiagnostics with the sensitivity of nucleic acid amplification-based diagnostics.

While this invention has been particularly shown and described with references to specific embodiments, it will be understood by those skilled in the art that various changes in form and details may be made therein without departing from the spirit and scope of the invention as defined by the appended claims.

1. A composition comprising a nucleic acid ligand affixed to a support, said nucleic acid ligand having affinity for prion protein.

2. The composition of claim 1 wherein said nucleic acid ligand is a RNA.

3. The composition of claim 2 wherein said RNA is RQ 11+12.

4. The composition of claim 1 wherein said support is selected from the group consisting of particles, beads, dipsticks, fibers, inner walls of containment vessels, thin-layer plates, membranes, and gels.

5. The composition of claim 1 wherein prion protein bound to said nucleic acid ligand is insoluble.

6. The composition of claim 1 wherein prion protein bound to said nucleic acid ligand is resistant to enzyme digestion.

7. The composition of claim 6 wherein said prion protein bound to said nucleic acid ligand is resistant to proteinase K digestion.

8. A device for isolating prion protein, comprising
a vessel for containing a sample and a support;
and a support, wherein said support has a nucleic acid ligand affixed thereto, said nucleic acid ligand having affinity to prion protein such that sample potentially containing prion protein may be placed in said vessel and said prion protein, if present, bind to said nucleic acid ligand.

9. The device of claim 8 wherein said vessel has an inlet and an outlet to allow sample to flow into said vessel through said inlet and allow said sample to flow out of said vessel through said outlet.

10. The device of claim 8 wherein said nucleic acid ligand is a RNA.

11. The device of claim 8 wherein said RNA is RQ 11+12.

12. The device of claim 8 wherein said support is selected from the group consisting of particles, beads, dipsticks, fibers, inner walls of containment vessels, thin-layer plates, membranes, and gels.

13. The device of claim 8 wherein prion protein bound to said nucleic acid ligand is insoluble.

14. The device of claim 13 wherein prion protein bound to said nucleic acid ligand is resistant to enzyme digestion.

15. The device of claim 14 wherein said prion protein bound to said nucleic acid ligand is resistant to proteinase K digestion.

16. A kit for concentrating prion protein in a sample, comprising a nucleic acid ligand affixed to a support, said nucleic acid ligand having affinity for prion protein.

17. The kit of claim 16 wherein said nucleic acid ligand is a RNA.

18. The kit of claim 16 wherein said RNA is RQ 11+12.

19. The kit of claim 16 wherein said support is selected from the group consisting of particles, beads, dipsticks, fibers, inner walls of containment vessels, thin-layer plates, membranes, and gels.

20. The kit of claim 16 wherein prion protein bound to said nucleic acid ligand is insoluble.

21. The kit of claim 20 wherein prion protein bound to said nucleic acid ligand is resistant to enzyme digestion.

22. The kit of claim 21 wherein said prion protein bound to said nucleic acid ligand is resistant to proteinase K digestion.

23. The kit of claim 16 further comprising one or more antibodies capable of binding an insoluble isoform.

24. The kit of claim 16 further comprising an enzyme capable of digesting a soluble isoform of the prion protein said kit for concentrating said prion protein on said support and subjecting said prion protein to digestion conditions with said enzyme to form a digestion product which in the event said prion protein is of the soluble form is digested and if such prion protein is of the insoluble form is not digested.

25. The kit of claim 24 further comprising at least one second ligand capable of binding said prion protein in an insoluble form, said second ligand placed in said digest product and allowed to form an affinity complex in the presence of said insoluble form of prion protein indicating its presence.

26. The kit of claim 25 wherein said second ligand is an antibody.

27. A kit of claim 16 wherein said support is contained in a vessel.

28. A method of concentrating prion protein comprising the steps of providing a nucleic acid ligand affixed to a support, said nucleic acid ligand having affinity for prion protein and placing a sample in contact with said support under conditions in which said nucleic acid and prion protein form an affinity complex.

29. The method of claim 28 wherein said nucleic acid ligand is a RNA.

30. The method of claim 28 wherein said RNA is RQ 11+12.

31. The method of claim 28 wherein said support is selected from the group consisting of particles, beads, dipsticks, fibers, inner walls of containment vessels, thin-layer plates, membranes, and gels.

32. The method of claim 28 wherein prion protein bound to said nucleic acid ligand is insoluble.

33. The method of claim 28 wherein prion protein bound to said nucleic acid ligand is resistant to enzyme digestion.

34. The method of claim 28 wherein said prion protein bound to said nucleic acid ligand is resistant to proteinase K digestion.


SEQUENCE LISTING (7 sequences)

SEQ ID NO: 1 - RNA, 197 nt, Artificial
  Synthetic
gggguuuccaaccggaauuugagggaugccuaggcaucccccgugcgucccuuuacgagggauugucgacucuagucgacgucugggcgaaaaauguacgagaggaccuuuucgguacagacgguaccugagggaugccuaggcauccccgcgcgccgguuucggaccuccagugcguguuaccgcacugucgaccc

SEQ ID NO: 2 - RNA, 29 nt, Artificial
  Synthetic
gggaauuugagggacgauggguaaguggg

SEQ ID NO: 3 - RNA, 56 nt, Artificial
  Synthetic
gggccccguaacuucgggagaaggggugcucuguuagggugcaagcccgagagagc

SEQ ID NO: 4 - RNA, 130 nt, Artificial
  Synthetic
ggguucauagccuauucggcuucgcgcaugggaauuugagggacgauggggaagugggagcgcguuuuaaaggaccuuuuucccucgcguagcuagcuacgcgaggugaccccccgaaggggggugcccc

SEQ ID NO: 5 - RNA, 117 nt, Artificial
  Synthetic
ggguucauagccuauucggcuucgcgccccugggguuugccucaggagcgcguuuuaaaggaccuuuuucccuugcguaguagcuacgcgaggugaccccccgaaggggggugcccc

SEQ ID NO: 6 - RNA, 118 nt, Artificial
  Synthetic
ggguucauagccuauucggcuucgcgcccguuuauaauacuuagugagcgcguuuuaaaggaccuuuuucccucgcguagcuagcuacgcgaggugaccccccgaaggggggugcccc

SEQ ID NO: 7 - RNA, 157 nt, Artificial
  Synthetic
gggguuucgaaccggaauuugagggaugccuaggcaucccccgugcgucccuuuacgagggauugucgacucuagaggauccgguaccugagggaugccuaggcauccccgcgcgccgguuucggaccuccagugcguguuaccgcacugucgaccc